(19) United States
(12) Patent Application Publication
(10) Pub. No.: US 2008/0269323 A1
(43) Pub. Date: Oct. 30, 2008

(54) USE OF 4-(NITROOXY)-BUTYL-(S)-2-(6-METHOXY-2-NAPHTHYL)-PROPANOATE FOR TREATING PAIN AND INFLAMMATION

(75) Inventors: Pottumarthi V. PRASAD, Highland Park, IL (US); Manlio Bolla, Grasse (FR); Marianna Armogida, Valbonne (FR)

Correspondence Address: ARENT FOX LLP, 1050 CONNECTICUT AVENUE, N.W., SUITE 400, WASHINGTON, DC, 20036, US

(73) Assignee: NICOX S.A., Sophia Antipolis (FR)

(21) Appl. No.: 12/107,961

(22) Filed: Apr. 23, 2008

Related U.S. Application Data
(60) Provisional application No. 60/907,970, filed on Apr. 25, 2007.

(51) Int. Cl. A61K 31/341; A61K 31/216; A61P 29/00; A61P 25/00

(52) U.S. Cl. 514/471; 514/534

(57) ABSTRACT

The present invention relates to the use of 4-(Nitrooxy)-butyl-(S)-2-(6-methoxy-2-naphthyl)-propanoate (naproxcinod) for treating pain and inflammation, in particular musculo-skeletal disorders, in patients with congestive heart failure, liver disease, cirrhosis, pre-existing renal disease, volume depletion, elderly with renal impairment, chronic renal failure or essential hypertension.

The present invention relates to the use of 4-(Nitrooxy)-butyl-(S)-2-(6-methoxy-2-naphthyl)-propanoate (naproxcinod) for treating pain and inflammation, in particular musculo-skeletal disorders, in patients with severe heart disease, liver disease, pre-existing renal disease, volume depletion, elderly with renal impairment.

The COX-inhibiting nitric oxide donors (CINODs) are a new therapeutic class designed for the treatment of acute and chronic pain. Naproxcinod is a nitric oxide (NO)-releasing derivative of naproxen with reduced gastrointestinal and cardiovascular toxicity. Naproxcinod is in Phase III clinical trials for treatment of signs or symptoms of osteo-arthrite.

Non-steroidal anti-inflammatory drugs (NSAIDs) are widely used to alleviate pain. While they are considered relatively safe for acute and short term use, there are well known adverse effects in chronic users.

Conventional NSAIDs have potentially important renal adverse effects (Whelton A, AM J Med 1999; 106:13-24).

The principal risk factors to develop nephrotoxicity are: male, age more than 65 years, presence of cardiovascular pathologies, high doses, recent hospitalization for non renal diseases and concomitant assumption of nephrotoxic drugs (Perez Gutthan S et al., Arch Int Med 1996; 156: 2433-9). 20% of patients with one or more of these risk factors could develop renal failure when treated with NSAIDs. A significant relationship between dose and time is reported in almost all cases (Perazzella M, Hosp Pract 2001; 36:43-56).

NSAIDs can induce two different forms of acute renal failure. Decreased prostaglandin synthesis can lead to reversible renal ischemia and haemodynamically-mediated acute renal failure (Perazzella M A, Eras J, Am J Kid Dis 2000; 35:937-40). The second form of acute renal failure is acute interstitial nephritis. In patients consuming excessive amount of NSAIDs over a period of several years, papillary necrosis can occur.

NSAIDs reduce renal perfusion through prostaglandins PGI₂, PGE₂ and PGD₂ inhibition with the clinical implications (Whelton A, AM J Med 1999; 106:13-24). Indeed prostaglandins regulate renal blood flow and electrolytes excretion in response to endogenous vasoconstrictors stimuli especially in elderly patients with hypovolemia and under treatment with diuretics (Clive D M, Stoff J S, N Engl J Med 1984; 310:563-72).

Administration of NSAIDs has been shown repeatedly to promote a sodium retention essentially during the first three days of administration. The NSAIDs' induced sodium retention may have several important clinical consequences, such as, blood pressure increasing in salt-sensitive subjects, peripheral edema and body weight increasing.

The sodium retention may decrease the natriuretic efficacy of drugs including diuretics such as furosemide and it can blunt the antihypertensive effect of thiazide. Moreover, it may be the cause of acute destabilizations of blood pressure in hypertensive patients or decompensations of heart function in patients with congestive heart failure.

It was thus an object of the present invention to provide an NSAID with less negative impact on renal function and particularly sodium retention, which can be used to treat pain in patients with congestive heart failure, cirrhosis, chronic renal failure or essential hypertension.

Since hypoxia of the renal medulla is a possible precursor of the onset of acute renal failure in humans, and the attenuation of human PGE₂ synthesis is considered partly responsible of the loss of ability to improve medullary oxygenation, the release of prostaglandins is particularly important in high risk patients including patients with severe heart disease, liver disease, pre-existing renal disease, volume depletion, elderly with renal impairment.

It has been so surprisingly found that naproxcinod maintains the oxygenation of renal medulla and therefore it results less nephrotoxic than naproxen.

Accordingly, the present invention relates to the use of a NO-releasing naproxen of formula (I):

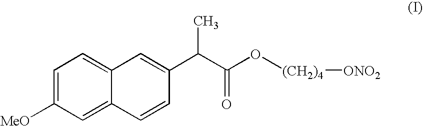

for treating pain and inflammation, in particular in musculo-skeletal disorders such as osteo-arthrite, in patients with congestive heart failure, liver disease, cirrhosis, pre-existing renal disease, volume depletion, elderly with renal impairment, chronic renal failure or essential hypertension. The compound is particularly useful in patients treated with diuretics such as furosemide and thiazides in general.

The doses to be administered are determined depending upon, for example, age, body weight, symptom, the desired therapeutic effect, the route of administration, and the duration of the treatment. In the human adult, the doses per person at a time are generally from 1 mg to 1000 mg, by oral administration, up to several times per day, and from 1 mg to 100 mg, by parenteral administration (preferably intravenous administration), up to several times per day, or continuous administration for from 1 to 24 hours.

As mentioned above, the doses to be used depend upon various conditions. Therefore, there are cases wherein doses lower than or greater than the ranges specified above may be used.

The compound of the present invention may be administered in the form of, for example, solid compositions, liquid compositions or other compositions for oral administration, injections, liniments or suppositories for parenteral administration.

The general synthesis of the NO-releasing drug of formula (I) is described in the WO95/09831.

### EXAMPLE 1

Effects of naproxcinod and naproxen on changes in medullary R₂* parameter, used as a semiquantitative measure of relative tissue oxigenation with Blood Oxigen Level Dependent Magnetic Resonance Imaging (BOLD-MRI) technique, were studied in rat kidneys.

The BOLD-MRI technique exploits the fact that the magnetic properties of hemoglobin vary depending on whether it is in the oxygenated or deoxygenated form. This affects the T₂* relaxation time of the neighboring water molecules and in turn influences the MRI signal on T₂*-weighted images. Because the ratio of oxyhemoglobin to deoxyhemoglobin is related to the pO₂ of blood, and since the pO₂ of capillary blood is thought to be in equilibrium with the surrounding tissue, changes estimated by BOLD-MRI can be interpreted as changes in tissue pO₂.

Eighteen male Sprague Dawley rats (315-320 g) were dosed orally by gavage with vehicle (carboxymethycellulose/DMSO), naproxcinod (14.5 mg/kg) or equimolar naproxen (10 mg/kg) for two weeks.

On the day of experiment, rats were anesthetized with Ketamine (60-100 mg/kg ip) and thiobutabarbital (100 mg/kg ip), catheterized in femoral vein and prepared for BOLD-MRI analysis. Technically, BOLD-MRI acquisitions were performed on a short bore Signa Twin speed 3.0T (GE Healthcare), using a multiple gradient echo sequence (TR/TE/Flip angle/FOV/BW/matrix/Thk/NXE=70 ms/4.4-57.7 ms/30°/10 cm/42 kHz/256×256/2 mm/10) to acquire sixteen T₂* weighted images.

A quadrature extremity coil was used for signal reception. The signal intensity vs. time data was fit to a single exponential function to generate R₂* map using the FUNCTOOL (GE Healthcare). The signal intensity vs. time data were fitted to a single decaying exponential function to determine the value of R₂*(=1/T₂*), that was used as a semiquantitative measure of relative tissue oxygenation. An increase in R₂* indicates a decrease in tissue pO₂.

After obtaining a set of baseline images, hypotonic glucose-saline (0.25% NaCl, 0.5% glucose) at 1.5 ml/100 g body weight/hr was infused intravenously via the femoral catheter for 2 hours to induce the water-diuresis.

R₂* maps were obtained every 3 minutes for 2 hours. Regions of interest (ROI) were placed on renal medulla to obtain values for the mean and standard deviation of R₂*. The statistical significance of the differences between pre- and post-diuresis R₂* was evaluated by two-tailed paired Student's t-test.

In control rats there was a significant shortening of R₂* which was completely abolished in the naproxen group, consistent with previous human findings. Surprisingly, in the naproxcinod group the response was almost intact (Tab. 1), even though the urinary PGE2 production levels were reduced in the naproxcinod group in a similar manner to that found for naproxen.

The urine flow rate increased in all groups during water-load (90 min) compared to baseline, but both naproxcinod and naproxen groups had substantially less increase in urine flow during water-load.

BOLD MRI observations during water-load clearly suggest differences in responses between naproxen and naproxcinod.

These results suggest that naproxcinod may have less nephrotoxicity in rats since it is less affecting renal medullary oxigenation.

TABLE 1
Normalized R₂* response in the cortex and medulla to
water-load in the three groups of animals

| | CORTEX | | | | | MEDULLA | | | | |
|---|---|---|---|---|---|---|---|---|---|---|
| | Time after the start of waterload (min.) | | | | | | | | | |
| Treatment | Basal | 30 | 60 | 90 | 120 | Basal | 30 | 60 | 90 | 120 |
| Vehicle | 100 | 98 | 96 | 95 | 94.5 | 100 | 95.5 | 91 | 86.5 | 85 |
| Naproxen | 100 | 102 | 101 | 100 | 96.5 | 100 | 103 | 103.5 | 102 | 101 |
| Naproxcinod | 100 | 99.5 | 97.5 | 97 | 98 | 100 | 94 | 90.5 | 86.5 | 89 |

1. A method for treating pain and/or inflammation in patients with congestive heart failure, liver disease, cirrhosis, pre-existing renal disease, volume depletion, elderly with renal impairment, chronic renal failure or essential hypertension, comprising administering a therapeutically effective amount of 4-(nitrooxy)-butyl-(S)-2-(6-methoxy-2-naphthyl)-propanoate.

2. The method of claim 1 wherein pain and inflammation are signs or symptoms of a musculo-skeletal disorders.

3. The method of claim 1 wherein pain and/or inflammation are signs or symptoms of osteo-arthrite.

4. The method of claim 1 wherein the patients are co-administered with a diuretic.

5. The method of claim 4 wherein the diuretic is furosemide and thiazides in general.
